(19) United States
(12) Patent Application Publication
(10) Pub. No.: US 2002/0120189 A1
(43) Pub. Date: Aug. 29, 2002

(54) Electromagnetic analyzer of anisotropy in chemical organized systems

(76) Inventor: Clarbruno Vedruccio, Medicina (IT)

Correspondence Address: LADAS & PARRY, 26 WEST 61ST STREET, NEW YORK, NY, 10023, US

(21) Appl. No.: 10/055,811

(22) Filed: Jan. 23, 2002

Related U.S. Application Data
(63) Continuation of application No. PCT/IT00/00316, filed on Jul. 26, 2000.

(30) Foreign Application Priority Data
Jul. 27, 1999 (IT) BO99A000422

(51) Int. Cl. A61B 5/05

(52) U.S. Cl. 600/407

(57) ABSTRACT

The present invention relates to an apparatus devoted to multiple use: preventive diagnostics in medical field, as the precocious diagnosis of anomalies of the woman breast, of the reproductive organs, and of many biological human and animal tissues anomalies. In the industry this invention could be used for analysis of agglomerations of materials like terrain, sand, concrete, tires, etc. and as detector, for security systems, of the crossing of areas what sheds, built, squares, open spaces, fluids also to low density and gas also extremely rarefied, and in the void. It can be used also for detection of buried metallic, conductive or dielectric objects of different composition from the analyzed terrain or generic area, operating on the specific compatible bands of frequency related to the matter to irradiate. Useful in archaeology, in techniques of geologic prospecting and many other fields like physics of the atmosphere, weather forecast systems, coherent multi-frequency oscillator in synthetic aperture radar (SAR), telecommunication devices and aerospace technologies.

### TECHNICAL FIELD

The present invention relates to an apparatus devoted to multiple use: preventive diagnostics in medical field, like the precocious diagnosis of anomalies of the woman breast, of the reproductive organs, and of many biological human and animal tissues anomalies. It is inserted, particularly, between the instruments that employ electromagnetic fields, of low intensity, for the diagnosis and the therapy of pathologies of various kind.

In the industry the present invention could be used for non destructive analysis of agglomerations of materials varied, like terrain, sand, concrete, tires, etc. and as detector, for security systems, of the presence and crossing of areas what sheds, built, squares, open spaces, fluids also to low density and gas also extremely rarefied, and in the void.

As detector for protection of external or inner areas, the system irradiates an electromagnetic field on the bands of biological interaction. Appropriate means of surveillance of the field, not exclusively connected via cable to the source, analyze the shift and the absorption of the bands of emitted frequency and, at the same time, elaborated by the coherent transceiver. For some particular applications, the probe can be installed in the focus of parabolic antennas or other suitable means to focalize the electromagnetic field, so as to be able to analyze perturbations of the far field, also at remarkable distances. The great advantage of the invention concerns the fact that is not possible to disturb or to cancel the operation of the instrument without allowing, the system for monitor, of the rf field to find anomalies and makes therefore to activate an alarm status. The said invention, as analyzer of anisotropy of materials of different kind and composition as an example bricks, tiles and similar, walls, land of varied composition, sandy shores, fluids, gases and in the vacuum. The said invention can be used also as detector of buried metallic, conductive or dielectric objects of different composition from the analyzed terrain or generic area, operating on the specific compatible bands of frequency related to the matter to irradiate. The said invention could have excellent performances also in archaeology, in techniques of geologic prospecting and many other fields like physics of the atmosphere, weather forecast systems, like multi band coherent local oscillator in innovative synthetic aperture radar (SAR), telecommunications devices and aerospace technologies.

The idea of solution of the present invention is that of to radiate an electromagnetic coherent energy, emitted by the hybrid state coherent transceiver probe, characterized by a multi-frequency pattern, toward the considered materials or the biologic tissues and to observe by means of an electromagnetic spectrum analyzer, the absorption lines and/or the frequency shift caused from the interaction with the structure under test. The same signals can also be evaluate with a digital computerized frequency meter, with selective voltmeters or specific radio receivers to record each variable correlated to the current analyses. For special purposes, related to the claims, it is also possible to demodulate the signals received and transmitted by the analyzer head. In this case the coherent multi frequency probe is directly connected to the demodulator and amplifier circuit, if or when installed, which provides further connections to oscilloscopes and other visualization means, and/or to suitable acoustic transducers.

### BACKGROUND ART

An extremely remarkable aspect to understand the potentialities of use of the said invention, for all that concerns the chemical organized systems, it is that this apparatus doesn't carry out investigations of structural determination by images, like for instance the X-ray Tomography, the Echography or the Nuclear Magnetic Resonance (NMR), but it analyze the functionality and the structural quality like variations of the electromagnetic pattern of the considered structures. The analysis is therefore possible simply sweeping the probe on the material to be analyzed or on the biological tissues, or vice versa. Regarding the resolution capabilities, or in other words the possibility to detect an anomalous tissue or structure, the precedent state of the art, related to the innovative microwave diagnostic equipment, can be made that, for ordinary system, they are able to identify among normal tissue those cancerous tissue particles which have a size larger than a quarter of the wavelength employed. This is not a problem for the said invention, as related in this descriptive text.

### DISCLOSURE OF INVENTION

Each complex structure is characterized by a state of specific order, that could be expressed in terms of entropic gradient. When the structure enters a state of alteration, in the case of living organisms, the complex mechanisms of feedback and regulation, in the necessity of compensate the altered status, employs energetic resources that are directly related to an increase of entropy of the considered structure.

This state of disorder could be revealed also, as an increase of the background noise of the electromagnetic activity; the structural alteration is characterized in the form of anisotropy of the considered whole, that could be easily detected by means of the coherent transceiver probe, core of the invention, and a spectrum analyzer or similar used as monitor.

The innovative aspect of the coherent transceiver probe is that it works in a reverse mode, i.e., it analyzes the continuum by tuning itself to the coherent vibration modes arising from complex long range EM processes, and detect the electromagnetic continuum anomaly or warping, regardless to the fractions of the wavelength used. To better explain the inventive concept, the coherent scanner, seems to be able to operate like a multi frequency, electromagnetic continuum isotropy analyzer, that could detect the energetic distribution pattern. The experiments recently carried out seems to support this statement.

At the current state of the research, for biomedical applications, the parameters analyzed by the instrument are normally related to the variations of amplitude of four lines of the spectral emission: the first line is relative to the structures characterized by an high content of calcium (bony structures etc.), she second spectral line furnishes data relative to the parenchyma and to the soft tissues, the third line responds to functional anomalies of the lynphatic and blood circulatory system.

The fourth spectral line, sometime not used, and characterized by an higher frequency, selectively responds to the ligaments structures and to the articulations cartilage; the observations till now implemented by Physicians suggests such comparison. The photos of the display of the spectrum analyzer furnish an exhaustive interpretative way. (FIGS. 1,2,3).

The general evaluation, during the screening, of the sudden reduction of the signal received by the spectrum analyzer, or in other words the attenuation of one or more spectral lines furnish the base for the diagnosis of the structures and/or of the irradiated tissues. The detectable data are immediate and of easy interpretation, so that they allow the Clinician to perform a rapid “total body” check of many states of alterations, also non symptomatic, of the subject under test. To acquire these data, the coherent transceiver probe, is swept on the surface of the body of the subject to be tested, without any need to remove his own clothes. The spectrum analyzer is normally at a distance of 1.5 or 2 meters to the patient, and receive the pre-elaborated data by means of a short antenna (r.f. sniffer) directly connected to the instrument input connector. This aspect gives, to the said invention, an extreme flexibility of use and a great portability, so as to be used by family doctors or anywhere a Diagnostic Center is not disposable or too far, like scientific researches missions in desolated lands, religious Missions or community in the world, aboard ships, aboard space station, (to evaluate the human body biophysical changes due to reduced gravity or cosmic radiation) etc.

As a result of various preliminary tests and experiments, the instrument showed to be able to find many states of alteration of the tissues, even preceding the clinical evidence of a pathology; and before they could be detected with the diagnostic equipment that the technology has offered in the last years. As an example, the said invention, appears particularly promising in the discovery of the biophysics alterations correlated to the appearance of many altered states of the health. This shows the capability of the invention in the field of the preventive functions. An interesting application of the said invention, could be in the area of legal medicine and the assurances medicine: the system is usefill, in connection to conventional systems, to the analysis of functionality of areas or tissues to prevent or to show swindles to the Insurance Companies.

### BRIEF DESCRIPTION OF DRAWINGS

FIGS. 1, 2, 3 are some spectral images, from the spectrum analyzer, of the signals emitted and elaborated by the coherent transceiver probe.

FIG. 4 represents the electric schematics diagram of the coherent transceiver.

FIG. 5 represents the block diagrams inside of the probe.

FIG. 6 Represents an alternative arrangements of the coherent transceiver.

### DETAILED DESCRIPTION OF THE INVENTION

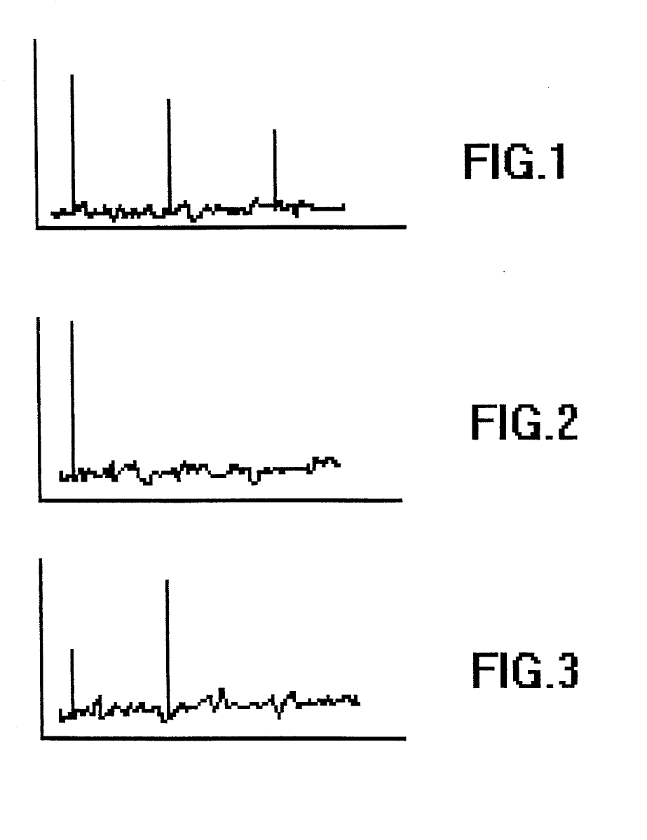

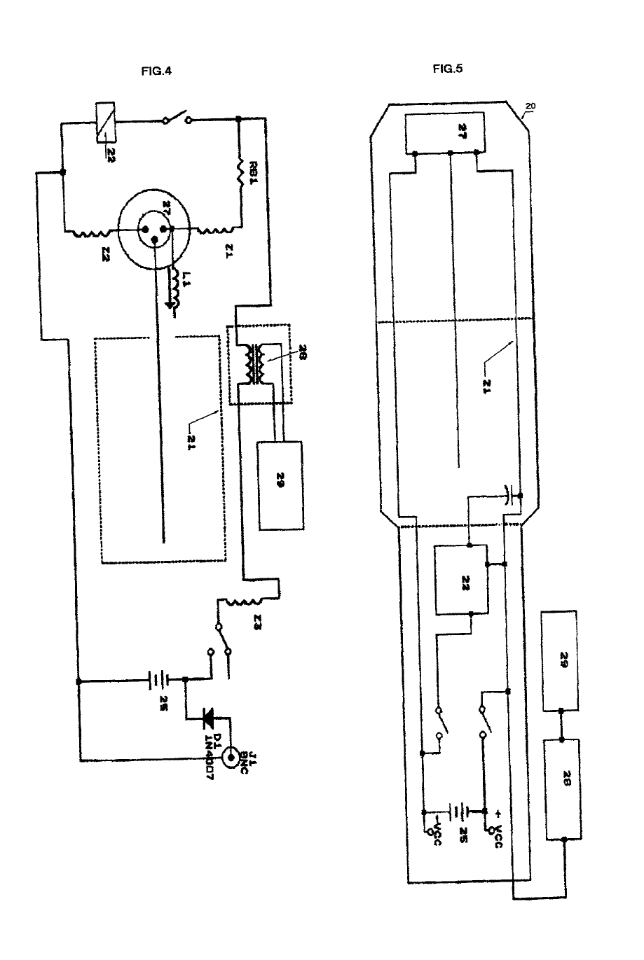

FIGS. 1-2-3 provides a not limiting example related to the bands of absorption of the field radiated by the coherent probe, towards a patient analyzed with such equipment. FIG. 1 shows the normal appearance of the lines on the spectrum analyzer display. FIG. 2 shows an example of a colon alteration (the second and third lines drops in the background noise) this means that there is an acute state of alteration of the colon tissue. FIG. 3 shows a cervical column alteration, characterized by an acute state (the first for bones, and third lines, related to the lynphatic system drops). A further application of the invention, is related to the possibility of therapy of altered conditions of the health, some of them, that could be treated with the normal electromagnetic therapy, others like Diabetes, Rheumatoid Arthritis, or problems related to weak vessel circulation, that could not be treated with normal electromagnetic therapy. The great innovative advantage, regarding the state of the technique, resides in the fact that the device is able to supply coherent electromagnetic energy selectively to the demand of the organism. In such a way the healing times are accelerated and, the amount of irradiated electromagnetic energy on the patient is dramatically reduced.

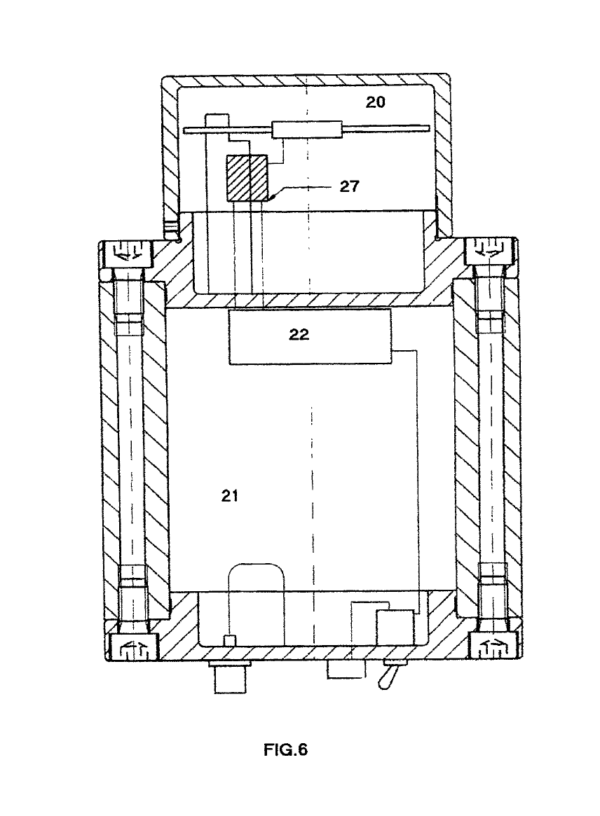

The technical characteristics of the invention, related to the aforesaid scopes, are clearly found from the written claims, and the advantages of the invention will turn out mainly obvious in the detailed description that it follows, made with reference to the attached designs, that represents a pure example and not limiting realization in which: FIG. 4 illustrates one block outline, not limiting, of an example of realization of the invention. FIG. 5 illustrates a schematic diagram of the electromagnetic generator in cavity, equipped of coherent oscillation module (27) and of the preliminary injection module (22) for the field generation, of its power supply (25), of ignition circuits, couplings and demodulator unit (28) the amplifier (29), contained in the handle (19), and of the exploring head dome (20).

As far as the not binding prototype of generator of coherent electromagnetic oscillations, the operation frequencies are comprised in the band of the 450-480 MHz and in its multiples that is 900, 1350, 1800 etc. with auto tuning possibility in plus or minus, for the detection of anisotropy of biological organisms, and comprised in whichever portion of the electromagnetic spectrum for tests on any organized chemical system, parameters that could be varied from a case to the other in function of the composition of the materials to analyze. The irradiated power from the invention is minimal, guaranteeing a low environmental, insignificant impact if compared in order of magnitude, to the field irradiated from a any cellular GSM telephone, (1 Watt vs. 1 mW of the said invention).

The power irradiated by the coherent transceiver probe is minimal, lower than 0 dBm@10 cm from the probe, that does not exclude however that could be manufactured a system, characterized by elevated powers for particular applications. The said invention, therefore is susceptible of numerous varying modifications, all re-entering in the within of the inventive concept. Moreover all the details can technically be replaced from equivalents elements.

1. Analyzer of anisotropy and entropy of organized chemical systems, characterized in that it comprises a coherent transceiver (21, 22, 27) of electromagnetic field, capable, when activated, to generate one or more spectral lines containing information concerning the interaction between the coherent electromagnetic beacon radiated by the coherent transceiver and the organized chemical system under test, said coherent transceiver being coupable to a spectrum analyzer for radio frequencies displaying said spectral lines to enable the analysis of the variation of the spectral lines and the evaluation of the states of structural anisotropy and entropy of the organized chemical system under test.

2. Analyzer of anisotropy and entropy of organized chemical systems according to claim 1, characterized in that it further comprises demodulation means coupled to said coherent transceiver (21, 22, 27) for demodulating the received signal.

3. Analyzer of anisotropy and entropy of organized chemical systems according to claim 1 or 2, characterized in that said coherent transceiver (21, 22, 27) radiate a coherent electromagnetic beacon on the bands of biological absorption

4. Analyzer of anisotropy and entropy of organized chemical systems according to any one of the preceding claims, characterized in that said coherent transceiver (21, 22, 27) comprises a cavity (21), and a coherent oscillating module (27) coupled to said cavity (21).

5. Analyzer of anisotropy and entropy of organized chemical systems according to claim 4, characterized in that said coherent transceiver (21, 22, 27) further comprises a module of preliminary injection of electromagnetic impulse (EMP) (22) coupled to said coherent oscillating module (27).

6. Method for analyzing anisotropy and entropy of organized chemical systems, characterized in that it comprises the steps of radiating an electromagnetic coherent energy towards the organized chemical system under test, and analyzing the absorption lines caused by the interaction of said electromagnetic coherent energy with the organized chemical system under test.
